(19) United States
(12) Patent Application Publication
(10) Pub. No.: US 2026/0102215 A1
(43) Pub. Date: Apr. 16, 2026

(54) SURGICAL ROBOT ARM EQUIPPED WITH SURGICAL INSTRUMENT AND METHOD OF OPERATING THE SAME

(71) Applicant: LIVSMED INC., Seongnam-si (KR)

(72) Inventors: Jin Hyuk YOON, Seongnam-si (KR); Young Jae SONG, Seongnam-si (KR); Jin Ah SIM, Yongin-si (KR); Se Ra KIM, Suwon-si (KR); So Yeon KIM, Yongin-si (KR)

(21) Appl. No.: 19/004,388

(22) Filed: Dec. 29, 2024

(30) Foreign Application Priority Data
Oct. 14, 2024 (KR) 10-2024-0139610

(51) Int. Cl. A61B 34/37; A61B 34/00; A61B 34/30

(52) U.S. Cl. CPC A61B 34/37; A61B 2034/302; A61B 2034/715

(57) ABSTRACT

Provided is a method of operating a surgical robot arm equipped with a surgical instrument, the method including acquiring ideal state change information of the surgical instrument corresponding to motion information of the surgical robot arm under a condition in which tension of a driving wire included in the surgical instrument is in a first state, acquiring actual state change information of the surgical instrument corresponding to the motion information under a condition in which the tension of the driving wire is not in the first state, calculating compensation motion information based on the ideal state change information and the actual state change information, and controlling the surgical instrument to operate based on the motion information comprising the compensation motion information.

### CROSS-REFERENCE TO RELATED APPLICATION

This application is based on and claims priority under 35 U.S.C. §119 to Korean Patent Application No. 10-2024-0139610, filed on Oct. 14, 2024, in the Korean Intellectual Property Office, the disclosure of which is incorporated by reference herein in its entirety.

### BACKGROUND

### 1. Field

The present disclosure relates to a surgical robot arm equipped with a surgical instrument and a method of operating the same.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like.

Accordingly, recently, surgery performed by inserting only a medical device, for example, laparoscopic surgical instrument, microsurgical microscope, and the like by forming a predetermined hole in the skin or surgery using a robot has been spotlighted as an alternative.

Here, a surgical robot refers to a robot that has a function of replacing a surgical action performed by a surgeon. Advantageously, the surgical robot may operate more accurately and precisely as compared with a human and enable remote operation.

Surgical robots that are currently being developed worldwide may include a bone surgical robot, a laparoscopic surgical robot, a stereotactic surgical robot, and the like. Here, the laparoscopic surgical robot is a robot that performs minimum invasive surgery using a laparoscope and small surgical instruments.

Laparoscopic surgery is a cutting-edge surgery technique that involves perforating one or more small holes in the abdomen and inserting a laparoscope, which is an endoscope for looking inside the abdomen to perform the surgery, and is a field that is expected to advance in the future. Today's laparoscopes are mounted with computer chips and have been developed to the extent that magnified images, which are clearer than images seen with the naked eye, can be obtained and when used with specially-designed laparoscopic surgical tools while looking at a monitor screen, any type of surgery is possible.

Moreover, laparoscopic surgery offers the same range of surgical procedures as open surgery, but with several advantages including fewer complications, the ability to initiate treatment shortly after the procedure, and the capability to maintain the patient's stamina and immune functions. As a result, laparoscopic surgery is becoming increasingly recognized as the standard surgery for treating colorectal cancer or the like in places such as the United States and Europe.

Meanwhile, a surgical robot is generally composed of a master robot and a slave robot. When a surgical operator manipulates a control lever (e.g., a handle) equipped on the master robot, a surgical tool coupled to or held by a robot arm on the slave robot may be manipulated to perform surgery.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

Some embodiments according to the present disclosure are directed to providing a surgical robot arm equipped with a surgical instrument and a method of operating the same. The problem to be solved by the present disclosure is not limited to the problems mentioned above, and other problems and advantages of the present disclosure, which are not mentioned, will be understood by the following description, and will be more clearly understood by the embodiments of the present disclosure. In addition, it will be appreciated that the problems and advantages to be solved by the present disclosure may be realized by means and combinations thereof indicated in the claims.

As a technical means for achieving the above technical object, a first aspect of the present disclosure may provide a method of operating a surgical robot arm equipped with a surgical instrument, the method including acquiring ideal state change information of the surgical instrument corresponding to motion information of the surgical robot arm under a condition in which tension of a driving wire included in the surgical instrument is in a first state, acquiring actual state change information of the surgical instrument corresponding to the motion information under a condition in which the tension of the driving wire is not in the first state, calculating compensation motion information based on the ideal state change information and the actual state change information, and controlling the surgical instrument to operate based on the motion information including the compensation motion information.

In the first aspect, the first state may include a state in which the driving wire transmits a predetermined percentage or more of power generated by the surgical robot arm to an end tool included in the surgical instrument.

In the first aspect, the acquiring of the ideal state change information may include acquiring a first initial state of the surgical instrument corresponding to an initial motion of the surgical robot arm, acquiring a first limit state of the surgical instrument corresponding to a limit motion of the surgical robot arm, and acquiring the ideal state change information based on the first initial state and the first limit state.

In the first aspect, the acquiring of the actual state change information may include acquiring a second initial state of the surgical instrument corresponding to an initial motion of the surgical robot arm, acquiring a second limit state of the surgical instrument corresponding to a limit motion of the surgical robot arm, and acquiring the actual state change information based on the second initial state and the second limit state.

In the first aspect, the ideal state change information and the actual state change information may include any one of a pulse value sensed by an encoder, a change in revolution number of a motor included in the surgical instrument, a change in rotational angle of the motor, and a change in torque of the motor.

In the first aspect, the calculating of the compensation motion information may include calculating the compensation motion information by combining a difference value between the ideal state change information and the actual state change information and a preset compensation coefficient.

In the first aspect, the controlling may include controlling an operation of the surgical instrument such that a portion of power generated by the surgical robot arm is used to increase the tension of the driving wire, and another portion of the power is used for an operation based on manipulation of a user.

In the first aspect, the acquiring of the first initial state may include acquiring a state at a time point at which the surgical robot arm is coupled to the surgical instrument.

In the first aspect, the acquiring of the first limit state may include acquiring a state of the surgical instrument at a time point at which an end tool included in the surgical instrument reaches a limit posture by the surgical robot arm.

In the first aspect, the acquiring of the ideal state change information may include acquiring the ideal state change information based on a difference between the first initial state and the first limit state.

In the first aspect, the acquiring of the second initial state may include acquiring a state at a time point at which the surgical robot arm is coupled to the surgical instrument.

In the first aspect, the acquiring of the second limit state may include acquiring a state of the surgical instrument at a time point at which an end tool included in the surgical instrument reaches a limit posture by the surgical robot arm.

In the first aspect, the acquiring of the actual state change information may include acquiring the actual state change information based on a difference between the second initial state and the second limit state.

A second aspect of the present disclosure may provide a surgical robot arm equipped with a surgical instrument, the surgical robot arm including at least one memory, and at least one processor, wherein the at least one processor acquires ideal state change information of the surgical instrument corresponding to motion information of the surgical robot arm under a condition in which tension of a driving wire included in the surgical instrument is in a first state, acquires actual state change information of the surgical instrument corresponding to the motion information under a condition in which the tension of the driving wire is not in the first state, calculates compensation motion information based on the ideal state change information and the actual state change information, and controls the surgical instrument to operate based on the motion information comprising the compensation motion information.

In the second aspect, the first state may include a state in which the driving wire transmits a predetermined percentage or more of power generated by the surgical robot arm to an end tool included in the surgical instrument.

In the second aspect, the at least one processor may acquire a first initial state of the surgical instrument corresponding to an initial motion of the surgical robot arm, may acquire a first limit state of the surgical instrument corresponding to a limit motion of the surgical robot arm, and may acquire ideal state change information based on the first initial state and the first limit state.

In the second aspect, the at least one processor may acquire a second initial state of the surgical instrument corresponding to an initial motion of the surgical robot arm, may acquire a second limit state of the surgical instrument corresponding to a limit motion of the surgical robot arm, and may acquire actual state change information based on the second initial state and the second limit state.

In the second aspect, the ideal state change information and the actual state change information may include any one of a pulse value sensed by an encoder, a change in revolution number of a motor included in the surgical instrument, a change in rotational angle of the motor, and a change in torque of the motor.

In the second aspect, the at least one processor may control an operation of the surgical instrument such that a portion of power generated by the surgical robot arm is used to increase the tension of the driving wire, and another portion of the power is used for an operation based on manipulation of a user.

A third aspect of the present disclosure may provide a computer-readable recording medium having recorded thereon a program for causing the method according to the first aspect to be executed on a computer.

In addition, other methods for implementing the present disclosure, other systems, and a computer-readable recording medium on which a computer program for executing the method is stored may be further provided.

Other aspects, features, and advantages other than those described above will become apparent from the following drawings, claims, and detailed description of the disclosure.

### BRIEF DESCRIPTION OF THE DRAWINGS

These and/or other aspects will become apparent and more readily appreciated from the following description of the embodiments, taken in conjunction with the accompanying drawings in which:

FIG. 1 is a conceptual diagram illustrating a surgical robot system equipped with a surgical instrument according to an embodiment of the present disclosure;

FIG. 2 is a block diagram illustrating an internal configuration of the surgical robot system of FIG. 1;

FIG. 3 is a perspective view illustrating a robot arm of the surgical robot system of FIG. 1 and a surgical instrument mounted thereon;

FIGS. 4 and 5 are diagrams illustrating a state in which the surgical instrument is mounted in FIG. 3;

FIG. 6 is a perspective view illustrating the surgical instrument according to an embodiment of the present disclosure;

FIG. 7 is a block diagram illustrating an example of an internal configuration of a surgical robot system according to an embodiment of the present disclosure;

FIG. 8 is a flowchart illustrating an example of a method of operating a robot arm according to an embodiment of the present disclosure;

FIG. 9 is a flowchart illustrating an example of a method of acquiring state change information according to an embodiment of the present disclosure;

FIG. 10 is a diagram for describing an example of a limit motion of a robot arm according to an embodiment of the present disclosure;

FIG. 11 is a block diagram illustrating an example of an internal configuration of a surgical robot arm according to an embodiment of the present disclosure; and

FIG. 12 is a flowchart illustrating an example of a method of operating a robot arm according to an embodiment of the present disclosure.

### DETAILED DESCRIPTION

The advantages and features of the present disclosure and methods of accomplishing the same will become apparent from the embodiments described in detail, taken in conjunction with the accompanying drawings. However, it should be understood that the present disclosure is not limited to the embodiments presented below, but may be implemented in various other forms and includes all transformations, equivalents, and substitutes included in the spirit and scope of the present disclosure. The embodiments presented below are provided so that this disclosure will be complete and will fully convey the scope of the invention to those skilled in the art. In describing the disclosure, when it is determined that the specific description of the known related art unnecessarily obscures the gist of the disclosure, the detailed description thereof will be omitted.

The terms used herein are merely used to describe specific embodiments and are not intended to limit the preset disclosure. An expression used in the singular encompasses the expression of the plural unless it has a clearly different meaning in the context. In the present application, the word “comprise” or “has” is used to specify existence of a feature, a numbers, a process, an operation, a constituent element, a part, or a combination thereof, and it will be understood that existence or additional possibility of one or more other features or numbers, processes, operations, constituent elements, parts, or combinations thereof are not excluded in advance.

Some embodiments of the present disclosure may be represented by functional block configurations and various processing operations. Some or all of the functional blocks may be implemented in various numbers of hardware and/or software configurations that perform particular functions. For example, the functional blocks of the present disclosure may be implemented by one or more microprocessors or by circuit configurations for a given function. Also, for example, the functional blocks of the present disclosure may be implemented in various programming or scripting languages. Functional blocks may be implemented as algorithms running on one or more processors. In addition, the present disclosure may adopt the related art for electronic configuration, signal processing, and/or data processing. Terms, such as “mechanism,” “element,” “means,” and “configuration” may be used widely and are not limited to mechanical and physical configurations.

In addition, the connecting lines or connecting members between the components shown in the drawings are merely illustrative of functional connections and/or physical or circuit connections. In a practical device, the connections between the components may be represented by various functional connections, physical connections, or circuit connections that may be replaced or added.

Hereinafter, embodiments will be described in detail with reference to the accompanying drawings. However, the embodiments may be implemented in various forms and are not limited to the examples described herein.

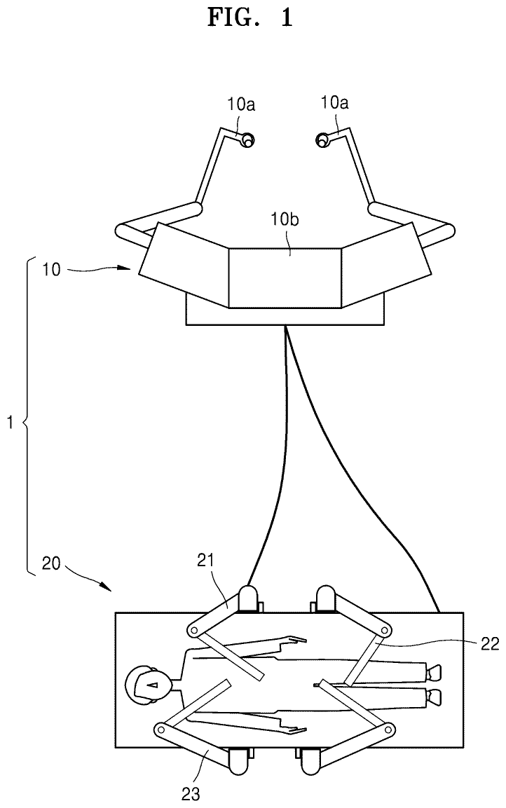

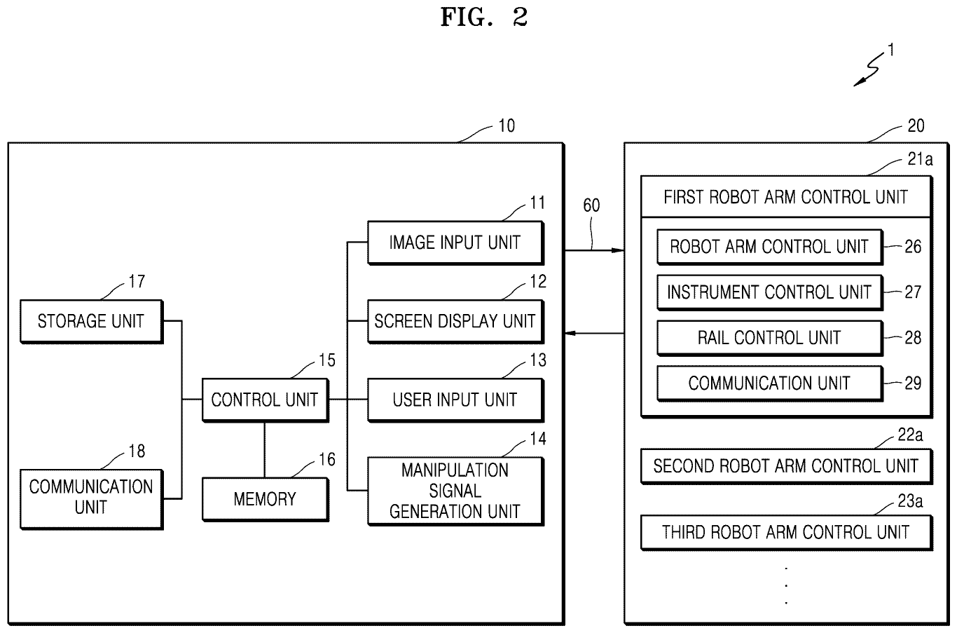

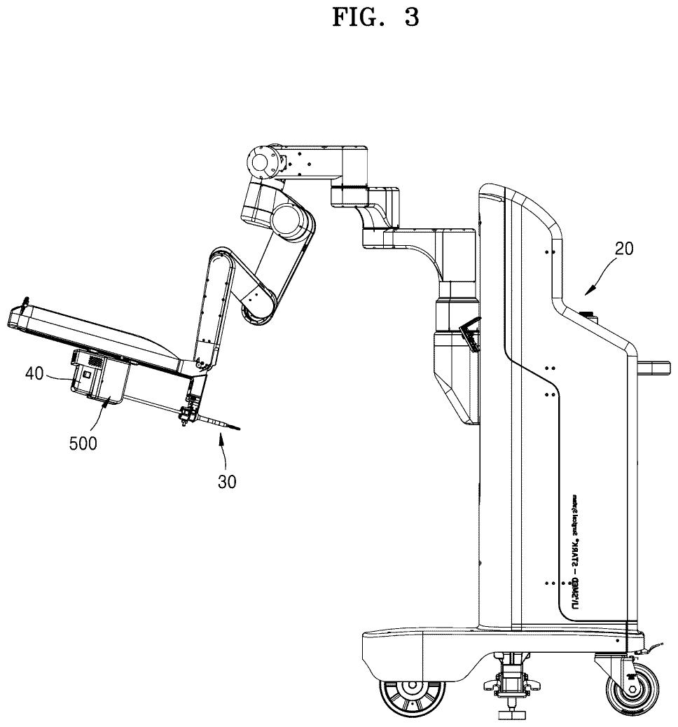

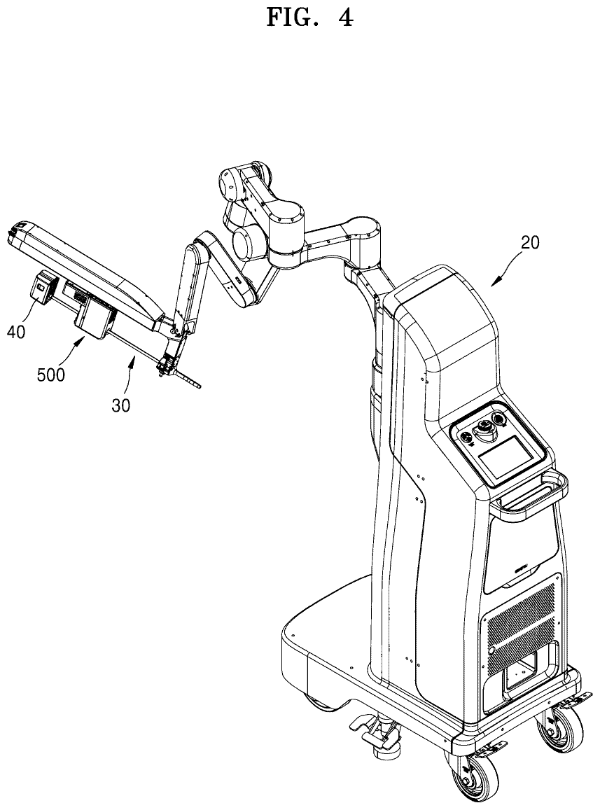

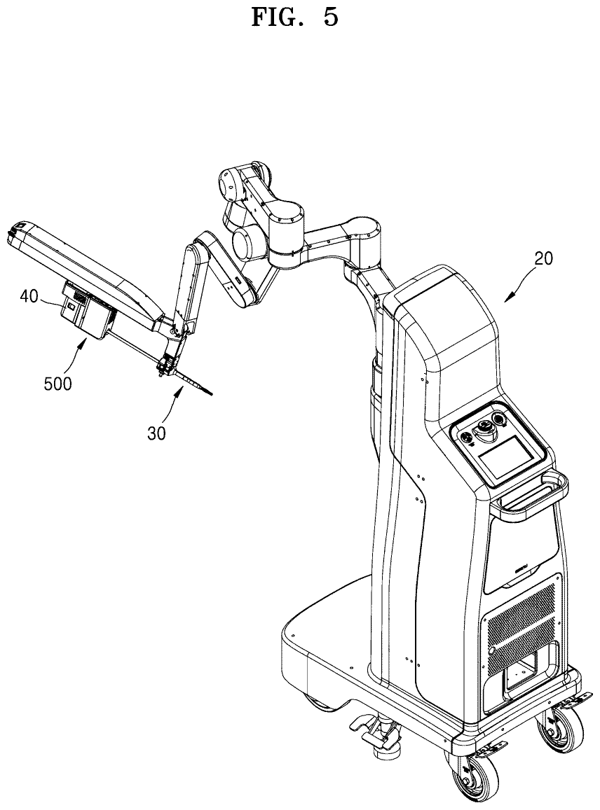

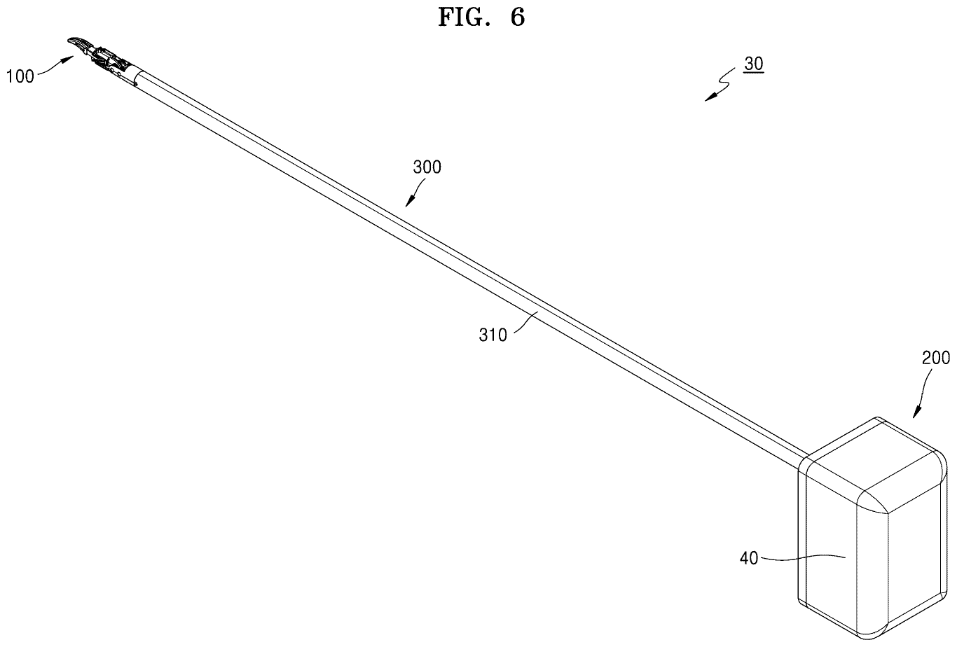

FIG. 1 is a conceptual diagram illustrating a surgical robot system 1 equipped with a surgical instrument according to an embodiment of the present disclosure. FIG. 2 is a block diagram illustrating an internal configuration of the surgical robot system 1 of FIG. 1. FIG. 3 is a perspective view illustrating a robot arm 20 of the surgical robot system 1 of FIG. 1 and a surgical instrument 30 mounted thereon. FIGS. 4 and 5 are diagrams illustrating a state in which the surgical instrument 30 is mounted in FIG. 3. FIG. 6 is a perspective view illustrating the surgical instrument 30 according to an embodiment of the present disclosure.

Referring to FIGS. 1 to 6, the surgical robot system 1 includes a master console 10, the robot arm 20, and the surgical instrument 30.

The master console 10 includes manipulation members 10a and a display member 10b, and the robot arm 20 includes one or more robot arm units 21, 22, and 23.

Specifically, the master console 10 includes the manipulation members 10a so that a surgical operator may grip and manipulate the manipulation members 10a with both hands. The manipulation members 10a may be implemented as two or more handles as shown in FIG. 1, and a manipulation signal according to the surgical operator's manipulation of the handles may be transmitted to the robot arm 20 through a wired or wireless communication network to control the robot arm units 21, 22, and 23. That is, surgical operations such as position movement, rotation, and cutting operations of the robot arm units 21, 22, and 23 may be performed by the surgical operator's manipulation of the handles.

For example, the surgical operator may manipulate the robot arm units 21, 22, and 23 using a handle-shaped manipulation lever. Such a manipulation lever may include various mechanical components according to manipulation methods thereof and may be provided in one of various forms for operating the robot arm units 21, 22, and 23 of the robot arm 20 and/or other surgical equipment, such as a master handle for manipulating the operations of the robot arm units 21, 22, and 23 and various input tools such as a joystick, a keypad, a trackball, a foot pedal, and a touch screen added to the master console 10 for manipulating a function of the entire system. Here, the manipulation member 10a is not limited to a shape of a handle, and any type may be applied without any limitation as long as the type may control the operations of the robot arm units 21, 22, and 23 through a network such as a wired or wireless communication network.

Alternatively, a voice input or motion input may also be applied for a user input. That is, a laparoscope (not shown in the drawing) included in the robot arm 20 may move in a direction in which a user wears glasses or a head mount display (HMD) with a sensor on the head and turns his/her gaze. Alternatively, when the user gives an instruction through a voice such as “left,” “right,” “first arm,” or “second arm,” the robot arm 20 may recognize the instruction and perform motions.

The display member 10b of the master console 10 displays an image captured through a laparoscope, which will be described below, as a screen image. In addition, a predetermine virtual manipulation plate may be displayed on the display member 10b together with the image captured through the laparoscope or independently. A detailed description of the arrangement, configuration, and the like of the virtual manipulation plate will be omitted.

Here, the display member 10b may consist of one or more monitors, and pieces of information necessary during surgery may be individually displayed on each monitor. The number of monitors may be variously determined according to the type or kind of information that needs to be displayed.

Meanwhile, the robot arm 20 may include one or more robot arm units 21, 22, and 23. Here, the robot arm units 21, 22, and 23 may be provided in module forms that may each independently operate, and in this case, an algorithm for preventing collision between the robot arm units 21, 22, and 23 may also be applied to the surgical robot system 1.

In general, a robot arm refers to a device that may have a similar function to a human arm and/or wrist and may attach a specific tool to a wrist area. In the present specification, the term “robot arm unit 21, 22, or 23” may be defined as a concept that includes all components such as the upper arm, lower arm, wrist, and elbow and a surgical instrument connected to a wrist area. Alternatively, the term “robot arm unit 21, 22, or 23” may be defined as a concept that includes only components for driving a surgical instrument excluding a surgical instrument that is coupled to a wrist area.

In this way, the robot arm units 21, 22, and 23 of the robot arm 20 may be implemented to have multiple degrees of freedom and be driven. The robot arm unit 21, 22, or 23 may include, for example, a surgical tool inserted into a surgical site of a patient, a yaw driving unit that rotates the surgical tool in a yaw direction according to a surgical position, a pitch driving unit that rotates the surgical tool in a pitch direction orthogonal to the rotational driving of the yaw driving unit, a transfer driving unit that moves the surgical tool in a longitudinal direction, a rotational driving unit that rotates the surgical tool, and a surgical tool driving unit that drives an end effector disposed at an end portion of the surgical tool to incise or cut a surgical lesion. However, the configuration of the robot arm units 21, 22, and 23 is not limited thereto, and it should be understood that such examples do not limit the scope of the present disclosure. Here, a detailed description of an actual control process in which the robot arm unit 21, 22, or 23 rotates or moves in a corresponding direction by a surgical operator manipulating the manipulation member 10a is omitted.

Here, the surgical instrument 30 may be attached to each of two of the robot arm units 21, 22, and 2, and a laparoscope may be attached to one thereof. A surgeon who is a surgical operator may select the robot arm units 21, 22, and 23, which are to be controlled, through the master console 10. In this way, the surgeon may directly control a total of three or more surgical tools through the master console 10 to accurately and freely control various tools according to the intention of the surgeon without a surgical assistant.

Meanwhile, one or more robot arms 20 may be provided to operate on a patient, and a laparoscope for displaying a surgical site as a screen image through the display member 10b may be implemented as an independent robot arm 20. In addition, as described above, the surgical robot system 1 of embodiments of the present disclosure may be universally used in surgeries that utilize various surgical endoscopes other than a laparoscope (for example, a thoracoscope, an arthroscope, a nasoscope, etc.).

Referring to FIG. 2, in an embodiment of the present disclosure, the master console 10 may include an image input unit 11, a screen display unit 12, a user input unit 13, a manipulation signal generation unit 14, a control unit 15, a memory 16, a storage unit 17, and a communication unit 18.

The image input unit 11 may receive an image captured by a camera provided on a laparoscope of the robot arm 20 through a wired or wireless communication network.

The screen display unit 12 outputs a screen image corresponding to the image received through the image input unit 11 as visual information. In addition, when a biometric information of a surgical patient is input, the screen display unit 12 may further output information corresponding to the biometric information. In addition, the screen display unit 12 may further output patient-related image data (for example, an X-ray image, a computerized tomography (CT) image, or a magnetic resonance imaging (MRI) image) about a surgical site. Here, the screen display unit 12 may be implemented in the form of the display member 10B (see FIG. 1), and an image processing process of outputting a received image as a screen image through the screen display unit 12 may be performed by the control unit 15.

In the embodiment illustrated in FIG. 2, the image input unit 11 and the screen display unit 12 are illustrated as being included in the master console 10, but one or more embodiments are not limited thereto. That is, the display member may be provided as a separate member separated from the master console 10. Alternatively, the display member may be provided as a component of the master console 10. In addition, in other embodiments, a plurality of display members may be provided, one thereof may be positioned adjacent to the master console 10, and some others thereof may be disposed at positions spaced a predetermined distance apart from the master console 10.

Here, the screen display unit 12 (that is, the display member 10B of FIG. 1) may be also provided as a stereoscopic display device. Specifically, a stereoscopic display device refers to an image display device in which a stereoscopic technology is applied to add depth information to a two-dimensional image, and the depth information is used to allow a viewer to feel a three-dimensional sense of liveliness and reality. The surgical robot system 1 according to an embodiment of the present disclosure may include the stereoscopic display device as the screen display unit 12 to provide a more realistic virtual environment to a user.

The user input unit 13 is a means for allowing a surgical operator to manipulate positions and functions of the robot arm units 21, 22, and 23 of the robot arm 20. The user input unit 13 may be formed in the form of the handle-shaped manipulation member 10A (see FIG. 1) as illustrated in FIG. 1, but a shape thereof is not limited thereto. The user input unit 13 may be modified and implemented in any one of various shapes to achieve the same purpose. In addition, for example, a portion of the user input unit 13 may be provided in the form of a handle, and the other portions thereof may be provided in different forms such as a clutch button. In addition, a finger insertion tube may be further formed to allow the surgical operator's finger to be inserted therethrough and fixed to facilitate manipulation of a surgical tool.

When a surgical operator manipulates the user input unit 13 to move the position of the robot arm unit 21, 22, or 23 or manipulate a surgical motion, the manipulation signal generation unit 14 generates and transmits a corresponding manipulation signal to the robot arm 20 through the communication unit 18. The manipulation signal may be transmitted or received through a wired or wireless communication network.

The control unit 15 is a type of central processing unit (CPU) that controls the operation of each component such that the above-described function may be performed. As an example, the control unit 15 may perform a function of converting an image input through the image input unit 11 into a screen image to be displayed through the screen display unit 12.

The memory 16 may perform a function of temporarily or permanently storing data processed by the control unit 15. Here, the memory 16 may include a magnetic storage medium or a flash storage medium, but the scope of the present disclosure is not limited thereto.

The storage unit 17 may store pieces of data received from the robot arm 20. In addition, the storage unit 17 may store various types of input data (for example, patient data, device data, and surgical data).

The communication unit 18 provides a communication interface necessary to transmit or receive image data transmitted from the robot arm 20 and control data transmitted from the master console 10 in conjunction with a communication network 60.

The robot arm 20 includes a plurality of robot arm unit control units 21a, 22a, and 23a. The robot arm unit control unit 21a includes a robot arm control unit 26, an instrument control unit 27, and a communication unit 29. In addition, the robot arm unit control unit 21a may further include a rail control unit 28.

Referring to FIGS. 2 and 3, the rail control unit 28 may control a movement path such that the surgical instrument 30 in the robot arm unit 21, 22, or 23 may move along a preset path, specifically, in a longitudinal direction of a power transmission unit 300.

The robot arm control unit 26 may receive a manipulation signal generated by the manipulation signal generation unit 14 of the master console 10 and may serve to control the robot arm units 21, 22, and 23 to operate according to the manipulation signal.

The instrument control unit 27 may receive the manipulation signal generated by the manipulation signal generation unit 14 of the master console 10 and may serve to control the surgical instrument 30 to operate according to the manipulation signal.

The communication unit 29 provides a communication interface necessary to transmit or receive image data transmitted from the robot arm 20 and control data transmitted from the master console 10 in conjunction with the communication network 60.

Meanwhile, the communication network 60 serves to connect the master console 10 and the robot arm 20. That is, the communication network 60 refers to a communication network that provides a connection path such that the master console 10 and the robot arm 20 may transmit or receive data to or from each other after being connected. The communication network 60 may include wired networks such as local area networks (LANs), wide area networks (WANs), metropolitan area networks (MANs), and integrated service digital networks (ISDNs), or wireless networks such as wireless LANs, code division multiple access (CDMA), Bluetooth, and satellite communications, but the scope of the present disclosure is not limited thereto.

Referring to FIGS. 3 to 6, the surgical instrument 30 to be described below may be connected to and installed on the robot arm unit 21, 22, or 23. An instrument case 40 may cover the surgical instrument 30 and may be connected to the robot arm unit 21. The instrument case 40 may cover one side of the surgical instrument 30 that is exposed to the outside, thereby blocking external foreign materials from reaching the surgical instrument 30 and preventing the surgical instrument 30 from being damaged by external impact.

A motor pack 500 may be connected to the surgical instrument 30 and may be coupled to the robot arm 20, specifically, the robot arm unit 21 so that a position thereof may be fixed.

The instrument case 40 may be connected to one side of the surgical instrument 30, and the motor pack 500 may be coupled to the other side opposite to the one side. The motor pack 500 may receive power from an external source to generate power, and the power generated by the motor pack 500 may be transmitted to the surgical instrument 30 so that the surgical instrument 30 may perform a pitch motion, a yaw motion, an actuation motion, and a roll motion.

Referring to FIG. 6, the surgical instrument 30 according to an embodiment of the present disclosure may include an end tool 100, a driving unit 200, and the power transmission unit 300, and the power transmission unit 300 may include a shaft 310.

Here, the shaft 310 may be formed in a shape of a hollow tube, and one or more driving wires (to be described below) may be accommodated inside the shaft 310. The driving unit 200 may be coupled to one end portion of the shaft 310, and the end tool 100 may be coupled to the other end portion opposite to the one end so that the shaft 310 may serve to connect the driving unit 200 and the end tool 100.

The driving unit 200 is formed at one end portion of the shaft 310 and provides an interface that may be coupled with the robot arm unit 21 (see FIG. 1). Accordingly, when the master console 10 (see FIG. 1) is operated by a user, the motor pack 500 connected to the robot arm unit 21 (see FIG. 1 or the like) may operate such that the end tool 100 of the surgical instrument 30 may perform a corresponding motion, and a driving force of the motor pack 500 may be transmitted to the end tool 100 through the driving unit 200. In other words, it may be described that the driving unit 200 itself serves as an interface connecting the surgical instrument 30 and the robot arm 20.

The end tool 100 is formed at the other end portion of the shaft 310 and is inserted into a surgical site to perform a motion necessary for surgery. As an example of such an end tool 100, a pair of jaws for performing a grip motion may be used. However, the idea of the present disclosure is not limited thereto, and various devices for surgery may be used as the end tool 100. For example, a component such as a one-armed cautery may also be used as the end tool 100.

The end tool 100 is connected to the driving unit 200 by the power transmission unit 300 and receives a driving force, which is generated by the motor pack 500 and transmitted to the driving unit 200, through the power transmission unit 300, thereby performing motions necessary for surgery, such as gripping, cutting, and suturing motions.

Here, the end tool 100 of the surgical instrument 30 according to an embodiment of the present disclosure may be formed to be rotatable in at least two directions, and for example, the end tool 100 may be formed to perform a pitch motion around a predetermined rotation axis and simultaneously perform a yaw motion and an actuation motion around another rotation axis.

The power transmission unit 300 may connect the driving unit 200 and the end tool 100, may serve to transmit a driving force of the driving unit 200 to the end tool 100, and may include a plurality of driving wires, pulleys, links, joints, gears, or the like.

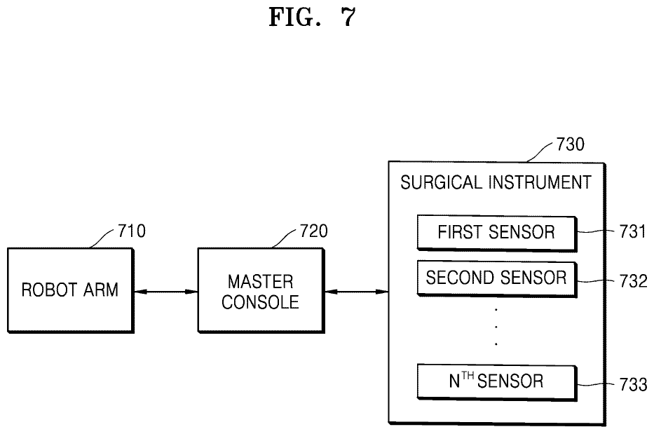

FIG. 7 is a block diagram illustrating an example of an internal configuration of a surgical robot system according to an embodiment of the present disclosure.

A process in which a surgical instrument is controlled by the manipulation of a surgical operator in the surgical robot system according to an embodiment of the present disclosure will be described with reference to FIG. 7.

Referring to FIG. 7, the surgical robot system according to an embodiment of the present disclosure may include a master console 710, a robot arm 720, and a surgical instrument 730. The master console 710, the robot arm 720, and the surgical instrument 730 may be identical to the master console 10, the robot arm 20, and the surgical instrument 30 described above with reference to FIGS. 1 to 6, respectively. Since a control flow of the surgical robot system is described with reference to FIG. 7, even when contents described above with reference to FIGS. 1 to 6 are omitted in FIG. 7, the contents may be equally applied to the surgical robot system of FIG. 7.

The master console 710 may receive the manipulation of a surgical operator. For example, the master console 710 may receive the manipulation of a user by using an manipulation member implemented in a shape of a handle.

The master console 710 may generate posture information about the robot arm 720 or the surgical instrument 730 based on the manipulation of the user. The posture information may be referred to as a target posture. For example, the posture information may include a target position, a target direction, or the like for the robot arm 720 or the surgical instrument 730. The master console 710 may transmit the posture information to the robot arm 720.

The robot arm 720 may generate motion information of the robot arm 720 or the surgical instrument 730 based on the posture information generated by the master console 710.

The motion information of the robot arm 720 may include, for example, a motion for changing a posture of a surgical tool. In an embodiment, the motion information of the robot arm 720 may include motions of one or more joints included in the robot arm 720 for changing a position of the surgical tool. In another embodiment, the motion information of the robot arm 720 may include motions of one or more joints included in the robot arm 720 for changing a direction of the surgical tool. In another aspect, the motion information of the robot arm 720 may be referred to as a motion of the joint included in the robot arm 720.

The motion information of the surgical instrument 730 may include, for example, a motion of an end tool included in the surgical instrument 730. In an embodiment, the motion information of the surgical instrument 730 may include a rotational motion of the end tool. Here, the rotational motion of the end tool may include roll rotation, pitch rotation, and yaw rotation. In another embodiment, the motion information of the surgical instrument 730 may include a motion of the end tool which is necessary for surgery. Here, the motion of the end tool may include a grip motion, a cutting motion, a suturing motion, and the like.

Meanwhile, in an embodiment, the motion information of the robot arm 720 may include a motion for controlling the surgical instrument 730 to operate. For example, the motion information of the robot arm 720 may include a motion for controlling the surgical instrument 730 to operate based on the generated motion information of the surgical instrument 730. As an example, the motion information of the robot arm 720 may include a motion for generating a signal for controlling the operation of the surgical instrument 730. As another example, the motion information of the robot arm 720 may include a motion for generating a driving force for the operation of the surgical instrument 730 and transmitting the generated driving force to the surgical instrument 730. Meanwhile, in an embodiment, a motion of the robot arm 720 for controlling the operation of the surgical instrument 730 may be performed irrespective of whether the master console 710 receives the manipulation of the user. For example, in order to calculate compensation motion information as described below, the robot arm 720 may perform an initial motion and a limit motion irrespective of posture information of the master console 710 and may acquire state change information of the surgical instrument 730. For example, the robot arm 720 may perform a series of motions to calculate compensation motion information in response to the surgical instrument 730 being mounted. For example, the robot arm 720 may perform the series of motions described above whenever a new surgical instrument 730 is mounted.

In summary, the robot arm 720 may operate based on the motion information of the robot arm 720 or may control the surgical instrument 730 to operate based on the motion information of the surgical instrument 730.

The surgical instrument 730 may operate in response to motion information generated by the robot arm 720.

In an embodiment, a motion of the surgical instrument 730 may include a motion for transmitting a driving force for the operation of the end tool. For example, the surgical instrument 730 may include a driving unit including one or more motors and a power transmission unit including one or more driving wires. For example, the motors included in the driving unit may include a motor for driving a rotary shaft connected to the end tool, a motor for driving a rotary shaft connected to a shaft, and the like. For example, a driving force generated by the robot arm 720 may be transmitted to the end tool through the motor included in the driving unit and the driving wire connected to the motor.

In an embodiment, the surgical instrument 730 may include a sensor unit that detects a state of the surgical instrument 730. For example, the sensor unit may include a plurality of sensors 731 to 733, and the plurality of sensors 731 to 733 may include a motor sensor capable of sensing various motor parameters such as a revolution number, a speed, a rotational direction, a rotational angle, and a rotational force (torque) of the motor included in the driving unit. For example, the motor sensor may be implemented as a motor encoder. As another example, the plurality of sensors 731 to 733 may include a motion sensor that detects movement of the surgical instrument 730. For example, the motion sensor may sense movement of the shaft or movement of the end tool.

Meanwhile, based on posture information generated by the master console 710 as described above, the robot arm 720 may generate motion information.

For example, the robot arm 720 may generate power for controlling the surgical instrument 730 to operate to a target posture. The power generated by the robot arm 720 may include power expected to be required for the surgical instrument 730 to reach the target posture, or power that should be continuously transmitted until the surgical instrument 730 reaches the target posture. For example, the robot arm 720 may transmit the generated power to the driving unit of the surgical instrument 730.

Here, in order for the power transmitted to the driving unit to be transmitted to the end tool without loss, tension having a predetermined magnitude or more needs to be applied to the driving wire. For example, when tension of the driving wire has a predetermined magnitude or more, the power transmitted to the driving unit may be used to operate the end tool, but when the tension of the driving wire has a magnitude that is less than the predetermined magnitude, a portion of the power transmitted to the driving unit may be used to increase the tension of the driving wire. As a result, power may be insufficient for the surgical instrument 730 to operate to the target posture, and more power may be required for the surgical instrument 730 to reach the target posture, or the surgical instrument 730 may perform only a posture change in response to the same power insufficient to reach the target posture.

In the present disclosure, there is provided a method of compensating for tension of the driving wire based on a change in state of the surgical instrument 730 corresponding to motion information of the robot arm 720.

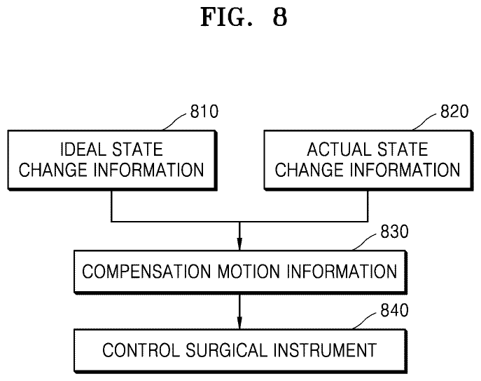

For example, in response to the motion information of the robot arm 720, the state of the surgical instrument 730 may change, and a change in the state may be detected by the sensor unit of the surgical instrument 730. The robot arm 720 may acquire state change information of the surgical instrument 730 and may calculate compensation motion information based on the state change information. Hereinafter, a detailed description will be given with reference to FIG. 8.

FIG. 8 is a flowchart illustrating an example of a method of operating a robot arm according to an embodiment of the present disclosure.

Referring to FIG. 8, in order to compensate for tension of a driving wire, a robot arm may acquire ideal state change information 810 and actual state change information 820 of a surgical instrument. As described above, the acquisition of the ideal state change information 810 and the actual state change information 820 of the surgical instrument by the robot arm may be performed irrespective of whether a master console receives the manipulation of a user. For example, the robot arm may acquire the ideal state change information 810 and the actual state change information 820 of the surgical instrument immediately after the surgical instrument is mounted or before the master console receives the manipulation of the user, and may generate compensation motion information based the ideal state change information 810 and the actual state change information 820. For example, after the robot arm generates the compensation motion information, motion information may be generated based on posture information of the master console, and the robot arm may control the operation of the robot arm or surgical instrument in consideration of both the compensation motion information and the motion information.

In an embodiment, the ideal state change information 810 of the surgical instrument may be state change information corresponding to motion information of a surgical robot arm under a condition in which the tension of the driving wire included in the surgical instrument is in a first state.

Here, the first state of the tension of the driving wire may refer to a tight state in which a driving force may be transmitted to an end tool without loss. For example, the tension of the driving wire may be in a tightest state at a time point at which the driving wire is included in the surgical instrument. For example, the first state of the tension of the driving wire may include an initial state before the surgical instrument is used for surgery.

As an example, in response to the surgical robot arm generating power for motion A and transmitting the power to the surgical instrument, an ideal state change of the surgical instrument may be a movement for performing motion A.

As another example, in response to the surgical robot arm controlling motion A of the end tool, the ideal state change of the surgical instrument may be a state change of a motor of the driving unit that transmits power for motion A of the end tool. For example, when a relationship between a state change of the motor of the driving unit and a motion of the end tool is known in advance, an ideal state change of the motor of the driving unit corresponding to motion A of the end tool may be determined in advance.

Meanwhile, in an embodiment, the actual state change information 820 of the surgical instrument may be state change information corresponding to motion information of the surgical robot arm under a condition in which the tension of the driving wire included in the surgical instrument is not in the first state. For example, a state in which the driving wire that is not in the first state may be refer to a loose state in which a portion of a driving force is used to increase tension.

As an example, in response to the surgical robot arm generating power for motion A and transmitting the power to the surgical instrument, an actual state change of the surgical instrument may be movement of the surgical instrument in which a portion of the power is used to increase tension, and the remaining power is used to perform motion A'. In an example, when it is assumed that an ideal state change of the surgical instrument corresponding to motion A of the surgical robot arm corresponds to motion A, an actual state change may correspond to motion A'.

As another example, in response to the surgical robot arm controlling motion A of the end tool, an actual state change of the surgical instrument may be a state change of the motor of the driving unit that transmits power for motion A of the end tool. For example, when it is assumed that the end tool of the surgical instrument requires a driving force with value A to perform motion A, an ideal state change of the surgical instrument may be a state change of the motor of the driving unit during a process of transmitting the driving force with value A. On the other hand, in addition to the driving force with value A, an actual state change of the surgical instrument may be a state change of the motor of the driving unit in a process of transmitting a driving force with value B which is used to increase tension of the driving wire. That is, an actual state change of the surgical instrument may be a state change of the motor of the driving unit in a process of transmitting a driving force with values A+B.

In an embodiment, the state change information of the surgical instrument may be a pulse value sensed by an encoder mounted on the driving unit. For example, the surgical robot arm may acquire the state change information of the surgical instrument based on the pulse value.

In an embodiment, the state change information of the surgical instrument may include a change in revolution number of the motor. For example, the surgical robot arm may acquire a revolution number of the motor that transmits a driving force to the end tool by using the driving wire. For example, the surgical robot arm may acquire the revolution number of the motor based on the pulse value sensed by the encoder.

In an embodiment, the state change information of the surgical instrument may include a change in rotational angle of the motor. As an example, the surgical robot arm may acquire the rotational angle of the motor, which transmits a driving force to the end tool by using the driving wire, based on a pulse value sensed by the encoder. As another example, the surgical robot arm may acquire the rotational angle based on a revolution number of the motor acquired from the encoder. For example, the rotational angle of the motor may be converted from the revolution number. For example, when the revolution number of the motor is 2, the rotational angle of the motor may be 720 degrees.

In an embodiment, the state change information of the surgical instrument may be a change in torque of the motor. For example, the surgical robot arm may acquire the change in torque of the motor that transmits a driving force to the end tool by using the driving wire. For example, the surgical robot arm may acquire the changes in torque of the motor from a motor sensor included in the driving unit.

Referring again to FIG. 8, the robot arm may calculate compensation motion information 830 based on the ideal state change information 810 and the actual state change information 820. For example, the compensation motion information 830 may be calculated based on a difference between the ideal state change information 810 and the actual state change information 820.

In an embodiment, the robot arm may calculate the compensation motion information 830 in further consideration of a compensation coefficient. For example, the robot arm may acquire the compensation motion information 830 by calculating a difference value between the ideal state change information 810 and the actual state change information 820 and then calculating the product of the difference value and the compensation coefficient. Here, as an example in which the compensation coefficient is determined, the compensation coefficient may be determined based on types of a surgical instrument or end tool. As another example, the compensation coefficient may be determined based on the usage environment of the surgical robot or surgical instrument. The compensation coefficient may be a value preset according to a used value or an experimental value by a surgical operator.

The robot arm may control the operation of the surgical instrument based on the compensation motion information 830. For example, after compensation for tension of the driving wire is completed, motion information of the robot arm for controlling the operation of the surgical instrument may include the compensation motion information 830 and existing motion information. For example, the robot arm may control the operation of the surgical instrument based on the existing motion information generated based on posture information of the master console and may also control the operation in further consideration of the compensation motion information.

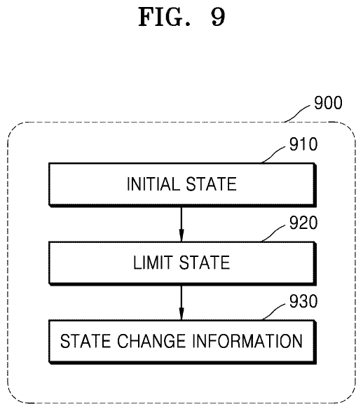

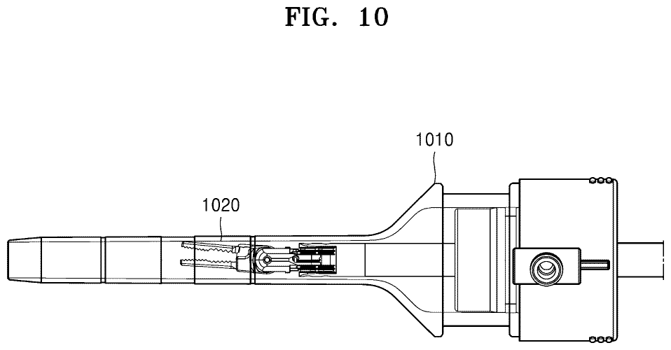

FIG. 9 is a flowchart illustrating an example of a method of acquiring state change information according to an embodiment of the present disclosure. FIG. 10 is a diagram for describing an example of a limit motion of a robot arm according to an embodiment of the present disclosure.

An example of a method of acquiring state change information of a surgical instrument will be described in detail with reference to FIG. 9.

Referring to FIG. 9, a method 900 of acquiring state change information according to an embodiment may include operations 910 to 930. For example, a surgical robot arm may acquire the ideal state change information 810 and actual state change information 820 of FIG. 8 through the method 900. In other words, operations 910 to 930 illustrated in FIG. 9 may be applied to a method of acquiring the ideal state change information 810 and a method of acquiring the actual state change information 820.

In an embodiment, the surgical robot arm may perform an initial motion and may acquire an initial state 910 of a surgical instrument corresponding to the initial motion.

For example, the initial motion of the surgical robot arm may be a motion for acquiring the initial state 910 of the surgical instrument. As an example, the initial motion may be a motion for acquiring an initial posture including an initial position and an initial direction of the surgical instrument. As another example, the initial motion may be a motion for acquiring an initial state of a motor included in a driving unit. For example, the initial state of the motor may include a state at a time point at which the surgical robot arm and the surgical instrument are mounted. For example, the surgical robot arm may perform a motion through which the surgical robot arm is coupled to the surgical instrument and may acquire a state of the motor at a time point at which the surgical robot arm is coupled to the surgical instrument. For example, the surgical robot arm may determine a time point at which torque of the motor changes abruptly as the time point at which the surgical robot arm is coupled to the surgical instrument. In an embodiment, the initial state of the motor may be understood as a reference for measuring a state change of the motor. As an example, when the state change of the motor is measured based on a pulse value, the initial state of the motor may be a pulse value sensed by an encoder at the time point at which the surgical robot arm is coupled to the surgical instrument. As another example, when the state change of the motor is measured based on a rotational angle of the motor, the initial state of the motor may be a rotational angle of the motor at the time point at which the surgical robot arm is coupled to the surgical instrument. In an embodiment, since the initial state 910 of the surgical instrument may be irrelevant to tension of a driving wire, a first initial state which is a criterion for an ideal state change may be the same as a second initial state which is a criterion for an actual state change.

In an embodiment, the surgical robot arm may perform a limit motion and may acquire a limit state 920 of the surgical instrument corresponding to the limit motion.

As an example, the limit motion of the surgical robot arm may include a motion for transmitting predetermined power. Here, the limit state 920 of the surgical instrument may be a state of the surgical instrument resulting from movement using the predetermined power. For example, the limit state 920 of the surgical instrument may be movement information of the surgical instrument using the predetermined power or posture change information of an end tool. For example, in response to reception of power A, a first limit state that is a criterion for an ideal state change may be a state in which the end tool rotates 30 degrees about a predetermined axis (for example, a pitch rotation axis), but a second limit state that is a criterion for an actual state change may be a state in which the end tool rotates 25 degrees about the predetermined axis (for example, the pitch rotation axis).

As another example, the limit motion of the surgical robot arm may include a motion for controlling the end tool included in the surgical instrument to a target posture. As an example, when the target posture of the end tool is generated based on the end tool rotating 30 degrees about the predetermined axis (for example, the pitch rotation axis), the limit motion of the surgical robot arm may refer to a motion for controlling the end tool to rotate 30 degrees about the predetermined axis.

As another example, the limit motion of the surgical robot arm may include a motion for controlling the end tool included in the surgical instrument to a limit posture. For example, the limit posture of the end tool may refer to a maximum posture to which the end tool or surgical instrument may be changed. For example, when a trocar 1010 is mounted on the surgical instrument as illustrated in FIG. 10, an end tool 1020 may operate inside the trocar 1010, and thus a limit posture of the end tool 1020 may refer to a position at which the end tool 1020 comes into contact with the trocar 1010. Meanwhile, the limit posture of the end tool is not limited thereto and may include all maximum postures to which the end tool may change due to physical constraints, mechanical limitations, design maximum values, or the like.

Here, the limit state 920 of the surgical instrument may be a state of the surgical instrument resulting from controlling the end tool to the limit posture. For example, the limit state 920 of the surgical instrument may be a state of the motor included in the driving unit. In another aspect, the limit state 920 of the surgical instrument may be a state of the surgical instrument at a time point at which the end tool reaches the limit posture. For example, the time point at which the end tool reaches the limit posture may be specified as a time point at which torque of the motor changes abruptly. For example, the limit state 920 of the surgical instrument may be a pulse value sensed by the encoder at the time point at which the torque of the motor changes abruptly.

For example, in response to controlling the end tool 1020 to change a posture to a position at which the end tool 100 comes into contact with the trocar 1010 as illustrated in FIG. 10, the first limit state that is the criterion for the ideal state change may correspond to three rotations of the motor, but the second limit state that is the criterion for the actual state change may correspond to four rotations of the motor. In addition, the limit state 920 of the surgical instrument may be measured by a pulse value sensed by the encoder as described above.

Referring again to FIG. 9, the surgical robot arm may acquire state change information 930 based on the initial state 910 and the limit state 920. For example, the surgical robot arm may acquire the state change information 930 based on a difference between the initial state 910 and the limit state 920.

For example, the surgical robot arm may acquire the state change information 930 based on a difference between a pulse value (initial state) sensed by the encoder at a time point at which the surgical robot arm is coupled to the surgical instrument and a pulse value (limit state) sensed by the encoder at a time point at which the end tool reaches the limit posture.

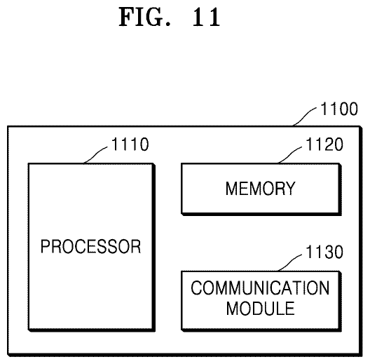

FIG. 11 is a block diagram illustrating an example of an internal configuration of a surgical robot arm 1100 according to an embodiment of the present disclosure.

Referring to FIG. 11, the surgical robot arm 1100 according to an embodiment may include a processor 1110, a memory 1120, and a communication module 1130. For convenience of description, only components related to the contents described with reference to FIGS. 7 to 10 are illustrated in FIG. 11. Accordingly, in addition to the components illustrated in FIG. 11, other components related to the contents described with reference to FIGS. 1 to 6 may be further included in the surgical robot arm 1100. In addition, it will be apparent to those skilled in the art related to the present disclosure that the processor 1110, the memory 1120, and the communication module 1130 illustrated in FIG. 11 may be implemented as independent devices.

The processor 110 may process instructions of a computer program by performing a basic arithmetic operation, a logic operation, and an input/output operation. Here, the instructions may be provided from the memory 1120 or an external device. In addition, the processor 1110 may control overall operations of the other components included in the surgical robot arm 1100.

For example, the processor 1110 may control at least some of operations of the surgical robot arm 1100 described above with reference to FIGS. 1 to 10. For example, the processor 1110 may control at least some of operations performed by the robot arm control unit or the instrument control unit of FIG. 2 or may control at least some of processes of the method of compensating for tension described with reference to FIGS. 7 to 10.

The processor 1110 may be implemented in an array of multiple logic gates, or in a combination of a universal microprocessor and a memory that stores a program executable in the microprocessor. For example, the processor 1110 may include a general-purpose processor, a CPU, a microprocessor, a digital signal processor (DSP), a controller, a microcontroller, a state machine, or the like. In some environments, the processor 1110 may include an application-specific semiconductor (ASIC), a programmable logic device (PLD), a field programmable gate array (FPGA), or the like. For example, the processor 1110 may refer to a combination of processing devices such as, for example, a combination of a DSP and a microprocessor, a combination of a plurality of microprocessors, a combination of one or more microprocessors in conjunction with a DSP core, or a combination of any other such configurations.

The memory 1120 may include any non-transitory computer-readable recording medium. As an example, the memory 1120 may include a permanent mass storage device such as a random access memory (RAM), a read-only memory (ROM), a disk drive, a solid state drive (SSD), or a flash memory. As another example, the permanent mass storage device such as a ROM, SSD, a flash memory, or a disk drive may be a separate permanent storage device which is distinguishable from the memory. In addition, an operating system (OS) and at least one program code (e.g., a code for the processor 110 to perform operations described above with reference to FIGS. 1 to 10) may be stored in the memory 1120.

These software components may be loaded from a computer-readable recording medium separate from the memory 1120. The separate computer-readable recording medium may be a recording medium that may be directly connected to the surgical robot arm 1100, and may include, for example, a computer-readable recording medium such as a floppy drive, a disk, a tape, a DVD/CD-ROM drive, or a memory card. Alternatively, the software components may be loaded into the memory 1120 through the communication module 1130 instead of the computer-readable recording medium. For example, at least one program may be loaded into the memory 1120 based on a computer program (e.g., a computer program for performing, by the processor 1110, the operations described with reference to FIGS. 1 to 10) installed by files provided through the communication module 1130 by developers or a computer file distribution system that distributes installation files of applications.

The communication module 1130 may provide a configuration or function for the surgical robot arm 1100 to communicate with an external device (for example, a master console or surgical instrument) through a network. For example, a control signal, a command, data, or the like, which is provided under the control of the processor 1110, may be transmitted to an external device through the communication module 1130 and the network.

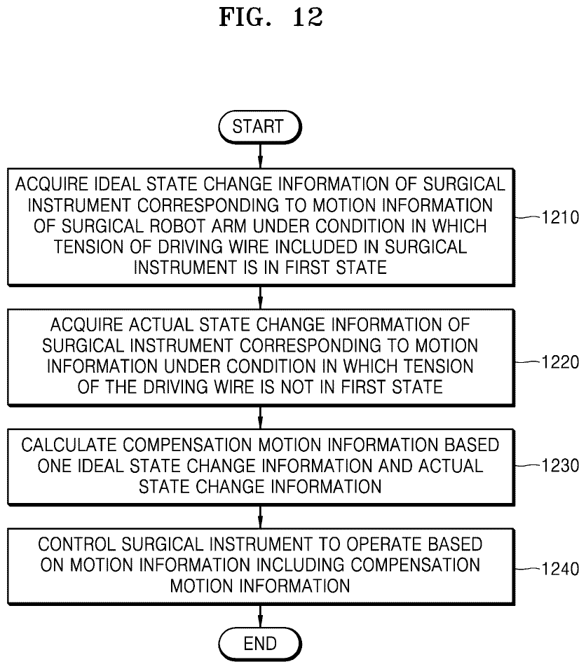

FIG. 12 is a flowchart illustrating an example of a method of operating a robot arm according to an embodiment of the present disclosure.

Referring to FIG. 12, the method of operating a robot arm according to an embodiment may include operations 1210 to 1240. For example, the operations illustrated in FIG. 12 may be executed by the processor 1110 described above.

First, in operation 1210, the processor 1110 may acquire ideal state change information of a surgical instrument corresponding to motion information of a surgical robot arm under a condition in which tension of a driving wire included in the surgical instrument is in a first state.

In an embodiment, the first state may be an initial state including a state set such that the driving wire transmits power generated by the surgical robot arm to an end tool included in the surgical instrument without loss.

In an embodiment, the processor 1110 may acquire a first initial state of the surgical instrument corresponding to an initial motion of the surgical robot arm, may acquire a first limit state of the surgical instrument corresponding to a limit motion of the surgical robot arm, and may acquire the ideal state change information based on the first initial state and the first limit state.

In operation 1220, the processor 1110 may acquire actual state change information of the surgical instrument corresponding to the motion information under a condition in which the tension of the driving wire is not in the first state.

In an embodiment, the processor 1110 may acquire a second initial state of the surgical instrument corresponding to the initial motion of the surgical robot arm, may acquire a second limit state of the surgical instrument corresponding to the limit motion of the surgical robot arm, and may acquire the actual state change information based on the second initial state and the second limit state.

In an embodiment, the ideal state change information and the actual state change information may include a change in revolution number of a motor included in the surgical instrument.

In another embodiment, the ideal state change information and the actual state change information may include a change in rotational angle of the motor included in the surgical instrument.

In another embodiment, the ideal state change information and the actual state change information may include a change in torque of the motor included in the surgical instrument.

In operation 1230, the processor 1110 may calculate compensation motion information based on the ideal state change information and the actual state change information.

In operation 1240, the processor 1110 may control the surgical instrument to operate based on the motion information including the compensation motion information.

Meanwhile, the above-described method may be recorded as a program that may be executed on a computer and may be implemented in a general-purpose digital computer operating the program using a computer-readable recording medium. In addition, the structure of the data used in the method described above may be recorded on a computer-readable recording medium through various means. Examples of the computer-readable recording medium include storage media such as magnetic storage media (e.g., a ROM, floppy disks, hard disks, and the like), and optical read media (e.g., CD-ROMs, DVDs, and the like).

Meanwhile, the above-described method may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices directly. When distributed online, at least a part of the computer program product may be temporarily generated or at least temporarily stored in the machine-readable storage medium, such as memory of the manufacturer's server, a server of the application store, or a relay server.

It will be understood by those skilled in the art to which the present embodiment pertains that the present disclosure may be implemented in modified forms without departing from the spirit and scope of the present disclosure. Therefore, the disclosed methods should be considered in an illustrative aspect rather than a restrictive aspect. The scope of the present disclosure should be defined by the claims rather than the above-mentioned description, and equivalents to the claims should be interpreted to fall within the present disclosure.

According to an embodiment of the present disclosure, tension of a wire included in a surgical instrument mounted on a surgical robot arm may be compensated for.

The effects of the present disclosure are not limited to those mentioned above, and other effects not mentioned may be clearly understood by those of ordinary skill in the art from the following description.

What is claimed is:

1. A method of operating a surgical robot arm equipped with a surgical instrument, the method comprising:
acquiring ideal state change information of the surgical instrument corresponding to motion information of the surgical robot arm under a condition in which tension of a driving wire included in the surgical instrument is in a first state;
acquiring actual state change information of the surgical instrument corresponding to the motion information under a condition in which a tension of the driving wire is not in the first state;
calculating compensation motion information based on the ideal state change information and the actual state change information; and
controlling the surgical instrument to operate based on the motion information comprising the compensation motion information.

2. The method of claim 1, wherein the first state comprises a state in which the driving wire transmits a predetermined percentage or more of power generated by the surgical robot arm to an end tool included in the surgical instrument.

3. The method of claim 1, wherein the acquiring of the ideal state change information comprises:
acquiring a first initial state of the surgical instrument corresponding to an initial motion of the surgical robot arm;
acquiring a first limit state of the surgical instrument corresponding to a limit motion of the surgical robot arm; and
acquiring the ideal state change information based on the first initial state and the first limit state.

4. The method of claim 1, wherein the acquiring of the actual state change information comprises:
acquiring a second initial state of the surgical instrument corresponding to an initial motion of the surgical robot arm;
acquiring a second limit state of the surgical instrument corresponding to a limit motion of the surgical robot arm; and
acquiring the actual state change information based on the second initial state and the second limit state.

5. The method of claim 1, wherein the ideal state change information and the actual state change information each comprise any one of a pulse value sensed by an encoder, a change in revolution number of a motor included in the surgical instrument, a change in rotational angle of the motor, and a change in torque of the motor.

6. The method of claim 1, wherein the calculating of the compensation motion information comprises calculating the compensation motion information by combining a difference value between the ideal state change information and the actual state change information and a preset compensation coefficient.

7. The method of claim 1, wherein the controlling comprises controlling an operation of the surgical instrument such that a portion of power generated by the surgical robot arm is used to increase the tension of the driving wire, and another portion of the power is used for an operation based on manipulation of a user.

8. The method of claim 3, wherein the acquiring of the first initial state comprises acquiring a state at a time point at which the surgical robot arm is coupled to the surgical instrument.

9. The method of claim 3, wherein the acquiring of the first limit state comprises acquiring a state of the surgical instrument at a time point at which an end tool included in the surgical instrument reaches a limit posture by the surgical robot arm.

10. The method of claim 3, wherein the acquiring of the ideal state change information comprises acquiring the ideal state change information based on a difference between the first initial state and the first limit state.

11. The method of claim 4, wherein the acquiring of the second initial state comprises acquiring a state at a time point at which the surgical robot arm is coupled to the surgical instrument.

12. The method of claim 4, wherein the acquiring of the second limit state comprises acquiring a state of the surgical instrument at a time point at which an end tool included in the surgical instrument reaches a limit posture by the surgical robot arm.

13. The method of claim 4, wherein the acquiring of the actual state change information comprises acquiring the actual state change information based on a difference between the second initial state and the second limit state.

14. A surgical robot arm equipped with a surgical instrument, the surgical robot arm comprising:
at least one memory; and
at least one processor;
wherein the at least one processor is further configured to
acquire ideal state change information of the surgical instrument corresponding to motion information of the surgical robot arm under a condition in which tension of a driving wire included in the surgical instrument is in a first state,
acquire actual state change information of the surgical instrument corresponding to the motion information under a condition in which the tension of the driving wire is not in the first state,
calculate compensation motion information based on the ideal state change information and the actual state change information, and
control the surgical instrument to operate based on the motion information comprising the compensation motion information.

15. The surgical robot arm of claim 14, wherein the first state comprises a state in which the driving wire transmits a predetermined percentage or more of power generated by the surgical robot arm to an end tool included in the surgical instrument.

16. The surgical robot arm of claim 14, wherein the at least one processor is further configured to
acquire a first initial state of the surgical instrument corresponding to an initial motion of the surgical robot arm,
acquire a first limit state of the surgical instrument corresponding to a limit motion of the surgical robot arm, and
acquire ideal state change information based on the first initial state and the first limit state.

17. The surgical robot arm of claim 14, wherein the at least one processor is further configured to
acquire a second initial state of the surgical instrument corresponding to an initial motion of the surgical robot arm,
acquire a second limit state of the surgical instrument corresponding to a limit motion of the surgical robot arm, and
acquire actual state change information based on the second initial state and the second limit state.

18. The surgical robot arm of claim 14, wherein the ideal state change information and the actual state change information each comprise any one of a pulse value sensed by an encoder, a change in revolution number of a motor included in the surgical instrument, a change in rotational angle of the motor, and a change in torque of the motor.

19. The surgical robot arm of claim 14, wherein the at least one processor is further configured to control an operation of the surgical instrument such that a portion of power generated by the surgical robot arm is used to increase the tension of the driving wire, and another portion of the power is used for an operation based on manipulation of a user.

20. A computer-readable recording medium having recorded thereon a program for causing the method of claim 1 to be executed on a computer.
